SEQUENCE LISTING (21 sequences)

SEQ ID NO: 1 - DNA, 20 bp, Artificial
  primer
cccgagtatctggaagacag

SEQ ID NO: 2 - DNA, 18 bp, Artificial
  primer
tatactcagagccggcct

SEQ ID NO: 3 - DNA, 20 bp, Artificial
  primer
ctatcaggacatagcgttgg

SEQ ID NO: 4 - DNA, 20 bp, Artificial
  primer
ttgcggcaagtttttggttg

SEQ ID NO: 5 - DNA, 22 bp, Artificial
  primer
agggcttctggttggctacaag

SEQ ID NO: 6 - DNA, 24 bp, Artificial
  primer
tgtctgtgtgtgctcctgaatcac

SEQ ID NO: 7 - DNA, 23 bp, Artificial
  primer
tggggtgggattagataaatgcc

SEQ ID NO: 8 - DNA, 20 bp, Artificial
  primer
ttttggttgtgggtctcctc

SEQ ID NO: 9 - DNA, 20 bp, Artificial
  primer
taggaattccaaggggtcaa

SEQ ID NO: 10 - DNA, 22 bp, Artificial
  primer
aacaaaaattcaaccaaacctc

SEQ ID NO: 11 - DNA, 22 bp, Artificial
  primer
tgtcttcattccagttgatgtg

SEQ ID NO: 12 - DNA, 24 bp, Artificial
  primer
aggctggagaggctgccaaggttt

SEQ ID NO: 13 - DNA, 23 bp, Artificial
  primer
acatggagtccgaagtgagcgag

SEQ ID NO: 14 - DNA, 26 bp, Artificial
  primer
ttcagcaagtgacttctgcgaagttc

SEQ ID NO: 15 - DNA, 23 bp, Artificial
  primer
ggccatgtggaccttgaggtgag

SEQ ID NO: 16 - DNA, 20 bp, Artificial
  primer
tgcctctctggagggcttcc

SEQ ID NO: 17 - DNA, 20 bp, Artificial
  primer
cagttcctcctgccagtgca

SEQ ID NO: 18 - DNA, 20 bp, Artificial
  primer
ttcgtgtgccattggggagg

SEQ ID NO: 19 - DNA, 20 bp, Artificial
  primer
cttgggctccactgtggaga

SEQ ID NO: 20 - DNA, 20 bp, Artificial
  primer
ttcgtgtgccgctggcaggc

SEQ ID NO: 21 - DNA, 20 bp, Artificial
  primer
ttgagcagtggagcacggac

(19) United States
(12) Patent Application Publication
(10) Pub. No.: US 2009/0094703 A1
(43) Pub. Date: Apr. 9, 2009

(54) Novel medulloblastoma-forming cell line

(75) Inventors: Peter Dirks, Toronto (CA); Ryan Ward, Toronto (CA); Chi-Chung Hui, Toronto (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(73) Assignee: The Hospital For Sick Children, Toronto (CA)

(21) Appl. No.: 12/230,809

(22) Filed: Sep. 5, 2008

Related U.S. Application Data
(60) Provisional application No. 60/935,960, filed on Sep. 7, 2007.

(51) Int. Cl. A01K 67/00; C12N 5/06; C12Q 1/02; G01N 33/00

(52) U.S. Cl. 800/3; 435/325; 800/13; 435/29

(57) ABSTRACT

Isolated medulloblastoma-forming clonogenic cells are provided which are useful to form stable cell lines as well as a non-human animal models of medulloblastoma that mimic human medulloblastoma, thereby providing a means to screen for candidate therapeutic compounds.

### FIELD OF THE INVENTION

The present invention generally relates to cells, cell lines and cell line culturing systems, and in particular, relates to novel medulloblastoma-forming cells.

### BACKGROUND OF THE INVENTION

Brain tumors are the second most common malignancy among children less than 20 years of age. Medulloblastoma is the most common malignant brain tumor arising in children, comprising 14.5% of newly diagnosed cases. Medulloblastoma originates in the cerebellum or posterior fossa and is the most common member of the family of cranial primitive neuroectodermal tumors (PNET). All PNET tumors of the brain are invasive and rapidly growing tumors that, unlike most brain tumors, spread through the cerebrospinal fluid (CSF) and frequently metastasize to different locations in the brain and spine.

Treatment of medulloblastoma includes surgical removal of tumour in combination with radiation and chemotherapy to increase the chances of disease-free survival. This combination may permit a 5 year survival in more than 80% of cases; however, aggressive treatment approaches, especially craniospinal irradiation, can harm the developing brain. It is hard to predict what dose of radiotherapy will be harmful in each individual child. On the other hand, the use of decreased dosages of radiation may not be sufficient to treat the tumour.

Over the past ten years, there has been increasing evidence that chemotherapy improves survival for children whose tumors cannot be fully resected, or have metastasized beyond the primary site at diagnosis. Studies attempting to delay and possibly even obviate radiotherapy in children, particularly those less than three years of age, are also underway using high-dose, multiple agent chemotherapy for administration immediately following surgery. Other studies are also underway involving chemotherapy directly within the cerebrospinal fluid.

Understanding the biology of medulloblastoma, its origin and what controls its growth is essential to the development of effective treatment protocols that obviate the disadvantages of current treatments, such as the adverse effects of radiation and side effects from non-specific treatments. It is known that human brain cancer growth is sustained by relatively rare therapy resistant cancer stem cells (CSC) both in vitro and in vivo. Although mouse tumours recapitulate many features of human cancer it is controversial whether these cancer models comprise a CSC hierarchy.

In order to advance the development of effective treatments for medulloblastoma, it would, thus, be desirable to develop a biological system which effectively mimics medulloblastoma in humans in order that novel therapies, such as chemotherapies, can be developed.

### SUMMARY OF THE INVENTION

Novel medulloblastoma-forming cells have now been identified which are clonogenic. The identification of such clonogenic cells permits the establishment of cell lines and non-human animal models that effectively mimic medulloblastoma in humans and, thus, which are useful to provide in vitro and in vivo screens for therapeutics to treat human medulloblastoma.

Thus, in one aspect of the present invention, isolated medulloblastoma-forming clonogenic cells are provided.

In another aspect, an isolated medulloblastoma-forming cell line is provided comprising Nestin+Sox2+ medulloblastoma cells.

In another aspect, a non-human transgenic animal model is provided that has been transplanted with a cell line comprising medulloblastoma-forming clonogenic cells.

In a further aspect, a method of producing a cell line comprising medulloblastoma-forming clonogenic cells. The method comprises the steps of obtaining a sample of medulloblastoma cells and culturing the medulloblastoma cells in serum free conditions with EGF and FGF to establish a cell line.

In yet a further aspect, methods of screening candidate therapeutic compounds both in vitro and in vivo are provided using the foregoing cell lines and transgenic animal model, respectively.

These and other aspects of the invention will become apparent in the following detailed description and the drawings in which:

### BRIEF DESCRIPTION OF THE DRAWINGS

FIG. 1 illustrates Ptc1 deficient medulloblastoma which are Nestin+;

FIG. 2 illustrates a method of obtaining Nestin+Sox2+ medulloblastoma cells from Ptc1 deficient medulloblastoma in vitro in serum free media containing EGF and FGF (a) capable of proliferation (b/c) but which stop proliferating in the presence of serum;

FIG. 3 illustrates the ptc genotype of the present Nestin+Sox2+ cells (a/b/c) and that these cells retain activated Hh and Notch signaling (d/f) and pharmacological sensitivities (e/f) in vitro;

FIG. 4 graphically illustrates inhibition of Hh signaling (a) and Notch signaling (b) in the present medulloblastoma cells;

FIG. 5 illustrates that Nestin+Sox2+ medulloblastoma cells initiate heterogeneous and representative tumour growth when transplanted into the cerebella of NOD/SCID recipients;

FIG. 6 graphically illustrates the colony-forming properties of various ptc genotypes;

FIG. 7 is a schematic illustrating ENU administration and CGCP proliferation in the developing mouse brain (a) and survival analysis of carcinogen treated pups up to six months of age (b); and

FIG. 8 illustrates the confluency in CD+ and CD− medulloblastoma cells.

### DETAILED DESCRIPTION OF THE INVENTION

Isolated medulloblastoma-forming clonogenic cells are provided that may be used to prepare cell lines and transgenic non-human animal models that mimic human medulloblastoma.

The term “isolated” is used herein to refer to medulloblastoma-forming clonogenic cells which are substantially free from non-proliferative medulloblastoma cells with which they exist in vivo, for example, substantially free from other cells of the primary tumor from which they were derived.

The term “clonogenic” is used herein to refer to cells which possess the potential to proliferate and form a colony of cells.

The medulloblastoma-forming clonogenic cells of the present invention are characterized as Nestin+Sox2+ cells that exhibit long term proliferative potential, multilineage differentiation capacity and retain activated Hedgehog (Hh) and Notch signaling as evidenced, for example, by established assay, e.g. assay for β-galactosidase expression.

In one embodiment, the medulloblastoma-forming clonogenic cells may be Patched1 (Ptc1)-deficient, e.g. exhibits deficient ptc1 expression as a result of the ptc1^{+/-} genotype. Patched1 is a receptor for Hedgehog signaling proteins. In another embodiment, the medulloblastoma-forming clonogenic cells may be deficient in tumour protein 53 (also known as protein 53 or p53), a tumour-suppressing transcription factor encoded by the TP53 gene. Tumour protein 53 (p53) deficiency refers to inclusion of either p53^{+/-} and p53^{-/-} alleles. In a further embodiment, the medulloblastoma-forming clonogenic cells are CD15^{+/-}, e.g. expresses CD15 (3-fucosyl-N-acetyl-lactosamine), also known as Lewis x and specific embryonic antigen 1 (SSEA-1), is a stem cell marker.

The medulloblastoma-forming clonogenic cell line in accordance with the present invention may be obtained using a culturing protocol in which Patched 1 (Ptc1)-deficient medulloblastoma cells are incubated in serum free conditions in the presence of epidermal growth factor (EGF) and fibroblast growth factor (FGF) for a period of time sufficient to result in enrichment of the clonogenic cells, for example, a period of time known in the art to be appropriate for cell culturing. The term “serum free” as used herein refers to the absence of any serous fluid including blood plasma. Using this protocol, a stable cell line is established which is viable through multiple passages, for example, at least 10 passages and preferably, greater than 25 passages, for example, greater than 40 passages.

An established medulloblastoma-forming cell line may be used in a method to screen candidate therapeutic compounds. Such a screening method includes the steps of culturing the cell line in the presence of a selected candidate compound for a sufficient period of time and then determining whether the candidate inhibits Hh or Notch signaling. This determination may be made by assaying the expression of Hh signaling targets, such as Gli1, Gli2 and/or Gli3, as well as the expression of Notch target genes such as Hes1 and Hes5. Decreased expression of an Hh or Notch target is indicative of inhibition of the Hh or Notch signaling pathway, respectively, in the cell line and indicative of the potential of the candidate as a therapeutic in the treatment of medulloblastoma.

Once obtained, a medulloblastoma-forming clonogenic cell line may be used to prepare a transgenic non-human animal model of medulloblastoma which mimics the disease in humans using conventional methodology. Methods of preparing transgenic animals are well-established in the art. In one embodiment, a method of preparing such an animal model includes injecting an amount of the cell line directly into the cerebella of the animal. Following a sufficient period of time, such as a period of at least about 5 weeks, and preferably a period of greater than 5 weeks, e.g. 8-12 weeks, evidence of medulloblastoma may be observed in the animal including ataxia, weight loss and an increase in intracranial pressure resulting in headaches, sickness (vomiting), sight problems, muscle weakness, fatigue and behavioural changes. Suitable animals for use in this aspect of the present invention may be any non-human mammal, including, without limitation, rodents such as mice, rats, hamsters, and gerbils, rabbits, cats, and dogs.

The non-human medulloblastoma animal model may also be used in a method of screening candidate compounds for therapeutic utility to treat medulloblastoma In particular, as the medulloblastoma animal model comprises the medulloblastoma-forming cells of the invention and these cells retain Hedgehog and Notch signaling activity in vitro, the animal model may be used to identify specific inhibitors that block these important developmental and cancer-associated pathways. A candidate compound may be administered to a medulloblastoma animal model, for example, by injection to the cerebellum or by systemic administration (oral, intravenous, intraperitoneal) before or after a tumor develops. The animal is then monitored for evidence that the candidate compound is inhibiting medulloblastoma formation or is causing medulloblastomas to reduce in size or disappear.

The present invention is described by reference to certain embodiments thereof; however, it will be understood by one of skill in the art that other embodiments of the invention as described and/or defined in the claims may be possible.

References noted herein are incorporated by reference.

Embodiments of the invention are described by reference to the following specific examples which are not to be construed as limiting.

### EXAMPLE 1

### Mouse Husbandry & Tissue Culture

C57/B6 Trp53^{+/-} mice (Jackson Laboratory, Maine, USA) and Ptc1^{+/-} mice were mated to generate a Ptc1^{+/-}p53^{+/-} breeding colony. Mice with symptoms of medulloblastoma, domed appearance of the head, unsteadiness of gait, were sacrificed by cervical dislocation. Brains were removed and tumours microdissected, dissociated by gentle pipetting in PBS and filtered through a 70 cm nylon filter. Cells were grown in serum free conditions with 20 ng/ml epidermal growth factor (EGF) and 20 ng/ml basic fibroblast growth factor (FGF) as previously described (Diamandis, P. et al. Nat Chem Biol 3, 268-73 (2007); Reynolds, B. A. & Weiss, S. Science 255, 1707-10 (1992)). For pharmacological studies, 5000 cells/well were grown in 96 well plates and treated with 5 μM cyclopamine (an inhibitor of Hh signaling) or 10 μM DAPT (an inhibitor of Notch signaling) for a week. 50 μl fresh media and inhibitor was added every second day. For differentiation analysis, cells were grown in DMEM/F12 media supplemented with 10% foetal bovine serum for 7 days. MTT assays were performed as previously described (Diamandis, P. et al. Nat Chem Biol 3, 268-73 (2007))

### Immunocytochemistry & Immunohistochemistry

Freshly dissociated tumour cells were cytospun onto glass slides (10⁵ cells/slide) and established cell lines were grown on glass coverslips. Cells were fixed in −20° C. methanol for 20 min. 4% paraformaldehyde fixed tissue was paraffin embedded and sectioned to generate 61 μm tissue slices. Cells and tissue sections were processed by standard protocol and stained with antibody as indicated in Table 1 below:

TABLE 1

| Antibody | Supplier | Purpose | Dilution & Condition |
|---|---|---|---|
| Mouse anti-Nestin | BD Biosciences, Canada | Immunocytochemistry | 1:500 1 hr RT |
| | | Immunohistochemistry | 1:125 O/N 4′ C. |
| Mouse anti-MAP2 | Chemion, USA | Immunocytochemistry | 1:125 1 hr RT |
| | | Immunohistochemistry | 1:100 O/N 4′ C. |
| Rabbit anti-Math1 | Biovision, USA | Immunohistochemistry | 1:100 O/N 4′ C. |
| Rabbit anti-Math1 | Abcam, USA | Immunocytochemistry | 1:125 1 hr RT |
| Rabbit anti-GFP | DakoCytomation, Denmark | Immunocytochemistry | 1:1000 1 hr RT |
| | | Immunohistochemistry | 1:500 O/N 4′ C. |
| Mouse anti-Tubulin, | Chemion, USA | Immunohistochemistry | 1:100 O/N 4′ C. |
| beta III | | | |
| Rabbit anit-Sox2 | Chemion, USA | Immunocytochemistry | 1:500 1 hr RT |
| | | Immunohistochemistry | 1:500 O/N 4′ C. |
| rat anti-Musashi, 14H1 | Dr. Okano, Japan | Immunocytochemistry | 1:250 1 hr RT |

Overnight: O/N
RT: Room Temperature

### Expression Analysis

Hh and Notch target genes were analyzed by RT-PCR and/or Western blot by standard procedures using the primers in the following Table 2. Where indicated, cells were analyzed after 7 days growth in the absence or presence of 5 μM cyclopamine or 10 μM DAPT.

TABLE 2

| | | |
|---|---|---|
| Primer Name | Primer Sequence | Purpose |

| | | |
|---|---|---|
| p53 Ex6 | CCC GAG TAT CTG GAA GAC AG | p53 genotyping¹ |
| p53 Ex7 | TAT ACT CAG AGC CGG CCT | p53 genotyping¹ |
| p53 Neo M5 | CTA TCA GGA CAT AGC GTT GG | p53 genotyping¹ |
| Ptc1 WT3 | TTG CGG CAA GTT TTT GGT TG | Ptc1 genotyping² |
| Ptc1 WT4 | AGG GCT TCT GGT TGG CTA CAA G | Ptc1 genotyping² |
| Ptc1Neo3 | TGT CTG TGT GTG CTC CTG AAT CAC | Ptc1 genotyping² |
| Ptc1 PT3 | TGG GGT GGG ATT AGA TAA ATG CC | Ptc1 genotyping² |
| Ptc1-Ex2-259 | TTT TGG TTG TGG GTC TCC TC | Ptc1 exon2 RT PCR |
| Ptc1-752 | TAG GAA TTC CAA GGG GTC AA | Ptc1 exon2 RT PCR |
| Ptc1-Ex4 F | AAC AAA AAT TCA ACC AAA CCT C | Ptc1 exon4 RT PCR² |
| Ptc1-Ex4 R | TGT CTT CAT TCC AGT TGA TGT G | Ptc1 exon4 RT PCR² |
| Hes1-F | AGG CTGG AGA GGC TGC CAA GGT TT | Hes1 RT PCR |
| Hes1-R | ACA TGG AGT CCG AAG TGA GCG AG | Hes1 RT PCR |
| Hes5-F | TTC AGC AAG TGA CTT CTG CGA AGT TC | Hes5 RT PCR |
| Hes5-R | GGC CAT GTG GAC CTT GAG GTG AG | Hes5 RT PCR |
| Ptc2 F | TGC CTC TCT GGA GGG CTT CC | Ptc2 RT PCR³ |
| Ptc2 R | CAG TTC CTC CTG CCA GTG CA | Ptc2 RT PCR³ |
| Gli1 F | TTC GTG TGC CAT TGG GGA GG | Gli1 RTPCR² |
| Gli1 R | CTT GGG CTC CAC TGT GGA GA | Gli1 RTPCR² |
| Gli2 F | TTC GTG TGC CGC TGG CAG GC | Gli2 RT PCR |
| Gli2 R | TTG AGC AGT GGA GCA CGG AC | Gli2 RT PCR |

1. Jacks, T. et al. Tumor spectrum analysis in p53-mutant mice. Curr Biol 4, 1-7 (1994).
2. Nieuwenhuis, E. et al. Mice with a targeted mutation of patched2 are viable but develop alopecia and epidermal hyperplasia. Mol Cell Biol 26, 6609-22 (2006).
3. Takabatake, T. et al. Hedgehog and patched gene expression in adult ocular tissues. FEBS Lett 410, 485-9 (1997).

### Orthotopic Injections

100,000 cells from established cell lines were suspended in approximately 2 μl cold PBS. NOD/SCID mice were prepared for surgery as previously described (Singh, S. K et al. Nature 432, 396-401 (2004)). Cells were injected into the right hemisphere of the cerebellum using a rodent stereotaxic headframe.

### Results

A phenotypic analysis of spontaneous medulloblastomas arising in mice deficient for one Ptc1 allele, alone or in combination with loss of the p53 tumor suppressor protein (Ptc1^{+/-}p53^{+/+}, Ptc1^{+/-}p53^{+/-}, or Ptc1^{+/-}p53^{-/-}), was performed. The expression of multiple neural lineages including MAP2+, NeuN+ and βIII-tubulin+ neurons, GFAP+ astrocytes and Math1+ GCPs was observed. In contrast to these abundant cell types, a subpopulation of Nestin+ and Sox2+ cells were detected, generally in close proximity to the vasculature: the putative CSC niche.

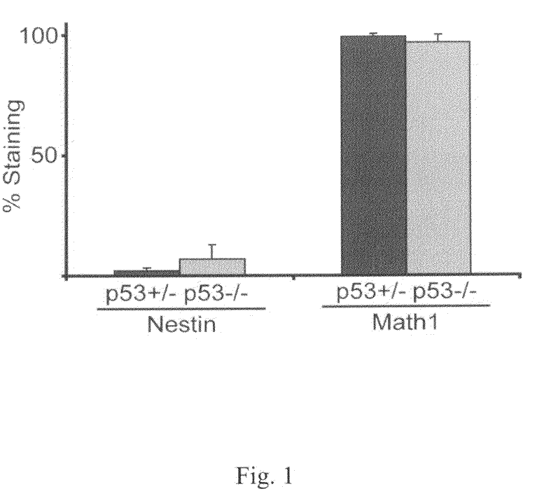

To better characterize the expression pattern of individual Ptc1^{+/-} MB cells, freshly dissociated tumor cells were stained for the markers observed in situ. While 96% of the cells expressed Math1, and a large proportion expressed GFAP or MAP2, a smaller proportion of cells were Nestin+ (FIG. 1). Consistent with studies of human medulloblastoma, these results demonstrate that Ptc1^{+/-} medulloblastoma comprise a heterogenous population of cells that express multiple differentiated and undifferentiated cell markers. This observation suggests the existence of a functional stem cell hierarchy in this phenotypically diverse population.

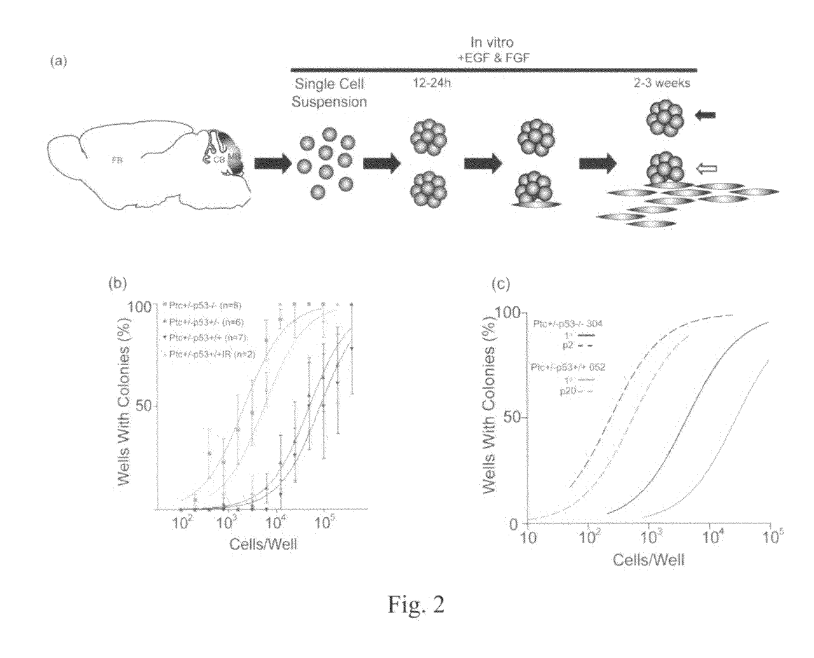

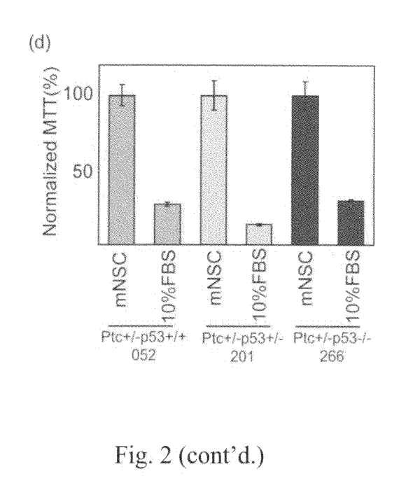

Cells were isolated from Ptc1^{+/-}p53^{+/+}, Ptc1^{+/-}p53^{+/-}, Ptc1^{+/-}p53^{-/-} and irradiated (IR) Ptc1^{+/-} medulloblastomas in vitro were then studied. Twenty-four hours after microdissection, dissociation and plating in serum free conditions (containing EGF and FGF), cells amalgamated into clusters, the majority of which did not go on to proliferate (FIG. 2a). One to three weeks later, adherent proliferating colonies were observed from every mouse medulloblastoma tumor tested, regardless of p53 genotype (FIG. 2b). In contrast, proliferating cells from normal, age-matched (>postnatal day 50) cerebellar tissue could not be cultured under identical conditions. Ptc1^{+/-} medulloblastoma cells primarily grew adherent to plastic, but occasionally as floating or semi-attached spheres, and could be expanded and propagated long term. X-gal staining demonstrated β-galactosidase expression from the mutant Ptc1 allele indicating a constitutively active Hh signaling pathway.

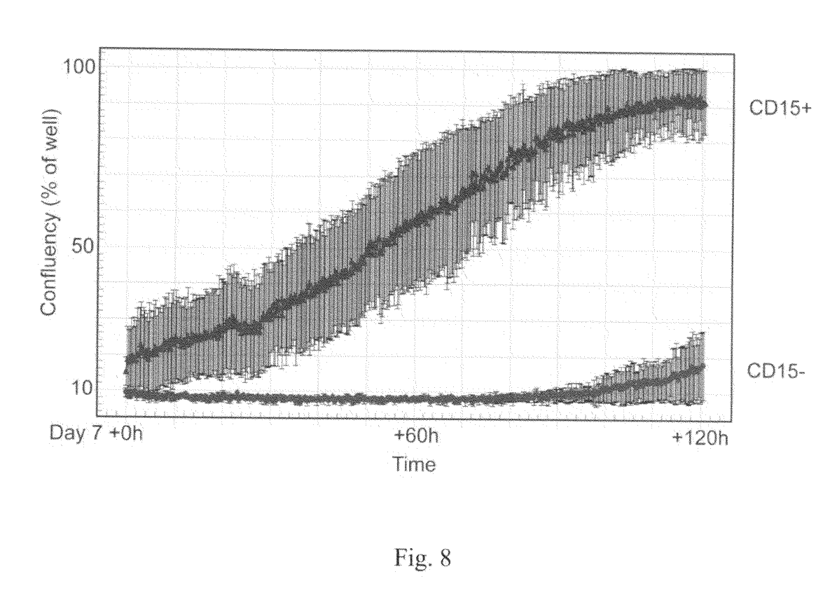

The clonogenic frequency of Ptc1^{+/-} medulloblastoma cells was determined by limiting dilution growth analysis (Tropepe, V. et al. Dev Biol 208, 166-88 (1999)) of freshly dissociated tumor cells (FIG. 2c). In all tumors studied, a subpopulation of cells capable of proliferating in serum free conditions were identified. The frequency of clonogenic cells was greater in Ptc1^{+/-}p53^{-/-} and Ptc1^{+/-} IR tumors compared to Ptc1^{+/-} p53^{+/+}πtumors, consistent with the increased aggressiveness and incidence of medulloblastoma in these mice. Once established, Ptc1^{+/-} medulloblastoma cells could be propagated long term (>50 passages) and were 50-fold enriched in clonogenic potential compared to the primary uncultured cells (FIG. 2c). Cells were Nestin+ and Sox2+. CD15+ cells were also determined to be enriched in clonogenic potential (FIG. 8). These cells also expressed Math1 and markers associated with the cerebellar stem cell, GFAP and Musashi, but not neuronal markers βIII tubulin or MAP2. When grown in the presence of 10% serum, Nestin+Sox2+ cells stopped proliferating (FIG. 2d) and adopted a large and flat morphology. Nestin expression was completely lost, but expression of Math1, GFAP and MAP2 was observed. Therefore, serum induces proliferative arrest and promotes differentiation of Nestin+Sox2+ cells. These results demonstrate that mouse medulloblastomas contain clonogenic cells with stem cell properties that are enriched by serum free culture conditions and can be induced to differentiate into multiple cell lineages.

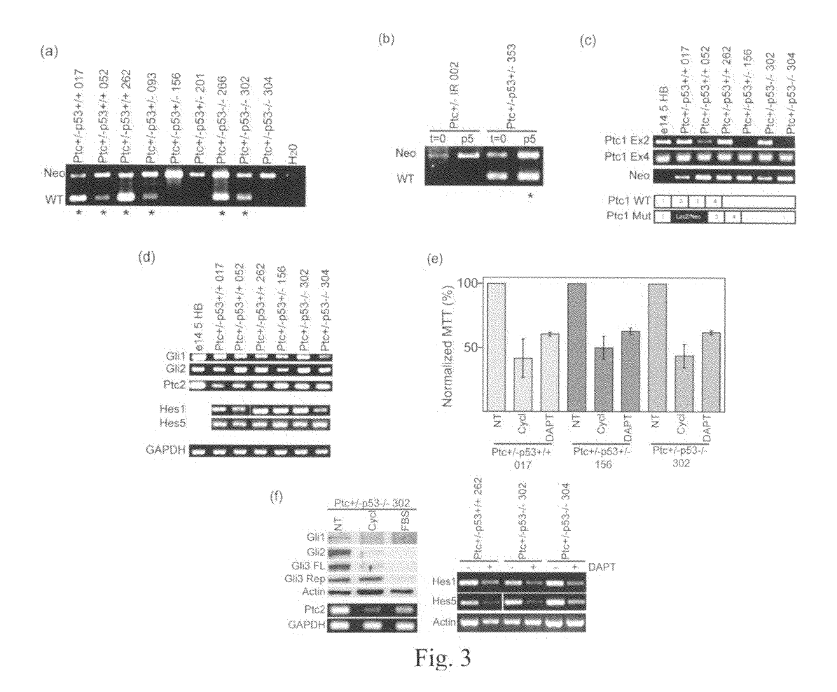

Loss of WT Ptc1 expression is thought to be a major contributing factor to tumor development in these mice. Medulloblastoma cell lines were genotyped, all of which originated from Ptc1^{+/-} mice, and a WT Ptc1 allele and mutant allele (marked by a Neo cassette) was detected in seven of 11 established, long-term cultured cell lines (FIG. 3a/b). Further, when passaged cell lines (p5) were analyzed, they showed the same Ptc1 genotype as the primary tumor from which they were derived (FIG. 3b, t=0). Premalignant and fully developed Ptc1^{+/-} medulloblastoma are thought not to express WT Ptc1 mRNA. However, in the cell lines that retained their WT Ptc1 allele, WT Ptc1 mRNA could be detected by RT-PCR analysis using exon2 specific primers (FIG. 3c). Embryonic neural precursor cells derived from WT hindbrain (e14.5 HB) were used as positive control for exons 2 and 4 expression and negative control for the neomycin resistance gene. These results indicate that loss of WT Ptc1 expression is not required for medulloblastoma development in these mice.

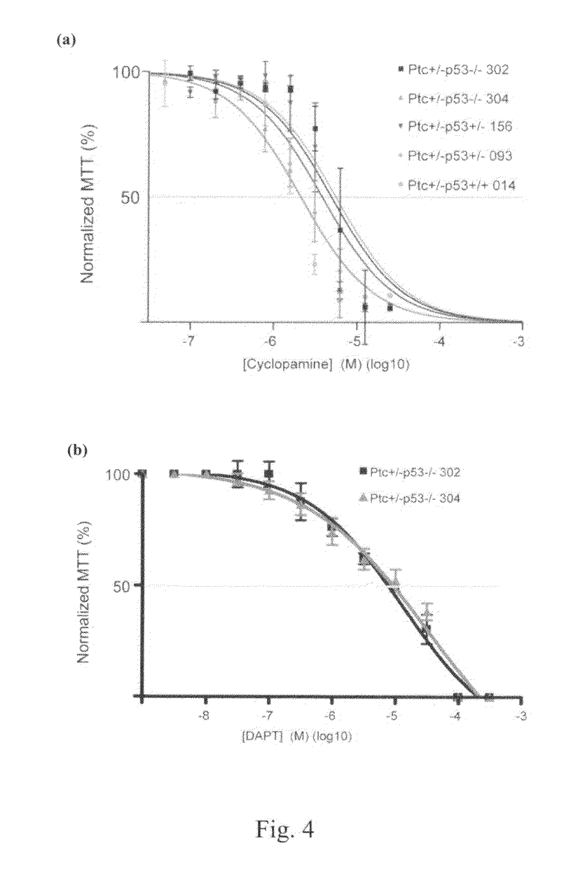

Ptc1^{+/-} medulloblastoma harbor activated Hh and Notch signaling in vivo, while serum derived Ptc1^{+/-} medulloblastoma cell lines do not and are thus a poor representation of the disease. The in vitro expression of Hh and Notch target genes in the cells were studied and their response to pharmacological pathway inhibitors were tested. Cell lines with differing Ptc1 and p53 genotypes (n=6) all demonstrated constitutively activated Hh and Notch signaling, expressing all Gli transcripts, Ptc2, Hes1 and Hes5 mRNA (FIG. 3d). Expression of Gli proteins was observed, for Gli3 in both full length (FL)/activator and repressor (Rep) forms (FIG. 3f). Importantly, cells responded to inhibitors of Hh (5 μM cyclopamine) and Notch (10M DAPT) signaling pathways (FIG. 3e/f and FIG. 4a/b). Cyclopamine treatment dramatically reduced protein expression of Hh signaling targets, Gli1 and Gli2, and increased the ratio of Gli3 in its repressor (Rep) form consistent with Hh signaling inhibition (FIG. 3f). DAPT treatment also resulted in downregulation of Notch target genes, Hes1 and Hes5. Strikingly, expression of all three Gli proteins was greatly reduced in serum-treated cells consistent with lack of activated Hh signaling in serum derived Ptc1^{+/-} medulloblastoma cell lines (FIG. 3f). The Ptc1^{+/-} medulloblastoma cell lines closely represent the original tumor as they maintain activated Hh and Notch signaling and sensitivity to specific pharmacological inhibitors in vitro as previously observed in vivo.

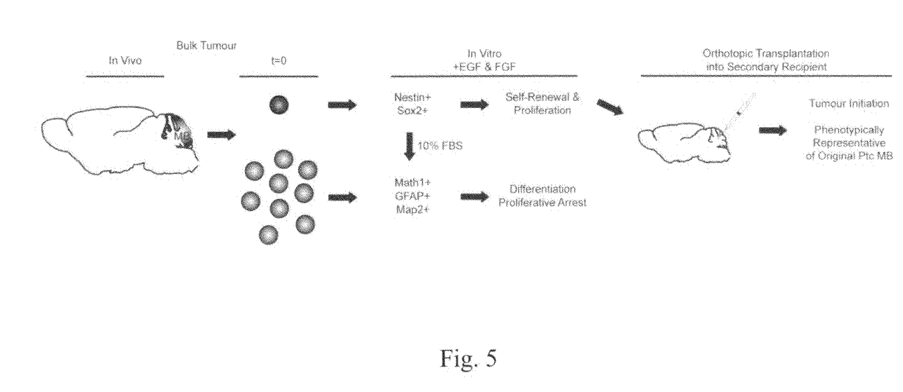

Finally, to determine the tumourigenic capacity of the Ptc1^{+/-} medulloblastoma cell lines, 10⁵ Nestin+Sox2+ cells were injected into the cerebella of NOD/SCID mice as shown in the schematic (FIG. 5). Eight to twelve weeks after injection mice displayed signs of ataxia, weight loss and intracranial pressure. Medulloblastoma were clearly apparent and resembled the primary tumor histology. Like primary Ptc1^{+/-} medulloblastoma sections, Nestin+ and Sox2+ cells were identified in the tumour grafts in close proximity to blood vessels. Virtually every tumor cell stained positive for Math1 and subpopulations GFAP and Map2 expressing cells were observed. This result demonstrates that the Nestin+Sox2+ medulloblastoma cells have the capacity to regenerate a heterogeneous and phenotypically representative Ptc1^{+/-} medulloblastoma in vivo.

### EXAMPLE 2

To determine if mouse medulloblastoma cells recapitulate a CSC hierarchy, an in vivo limiting dilution analysis (Goodrich et al. Science 277, 1109-1113 (1997)) of freshly isolated tumor cells derived from spontaneous medulloblastomas from Ptc1^{+/-} mice was performed alone or in combination with p53 deficiency (Ptc1^{+/-}p53^{+/+} or Ptc1^{+/-}p53^{-/-}) as set out in Table 3.

TABLE 3

| | # Cells Injected: | | | | |
|---|---|---|---|---|---|
| | 5 × 10⁵ | 1 × 10⁵ | 5 × 10⁴ | 1 × 10⁴ | 1 × 10³ |
| MB Genotype: | Tumor Initiation: | | | | |
| Ptc+/−p53+/+, − | 2/8 | 1/8 | — | 0/3 | — |
| Ptc+/−p53−/− | 2/3 | 1/2 | 3/4 | 0/2 | 0/2 |

Following orthotopic transplantation of cells into the cerebella of NOD/SCID mice, it was determined that injecting ≧5×10⁴ Ptc1^{+/-}p53^{-/-} medulloblastoma cells, but not ≦1×10⁴ cells, consistently generated medulloblastoma in recipient mice.

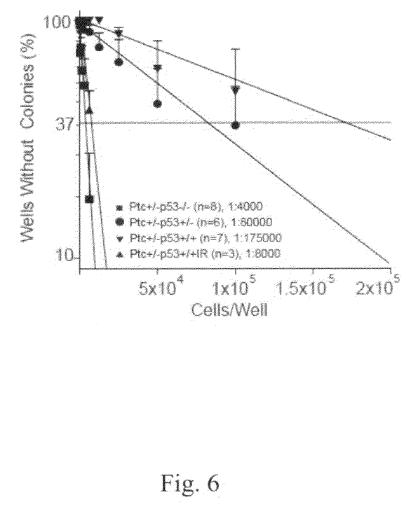

Fluorescence-activated cell sorting (FACS) of freshly dissociated, uncultured Ptc1^{+/-} medulloblastoma cells for CD15/Lewis X/stage specific embryonic antigen 1 (SSEA-1), a cell surface marker of embryonic and adult mouse neural precursor cells, was then performed. The abilities of CD15⁺ cells (representing 10-30% of the medulloblastoma cells) and CD15⁻ cells to initiate tumors after orthotopic injection was compared. The results are set out in Table 4 and as illustrated in FIG. 6.

TABLE 4

| | | | | | Tumor |
|---|---|---|---|---|---|
| MB Genotype | Tumor ID | Population | Purity | # | Initiation |
| Ptc+/−p53+/+ | 314 | CD15+ | 93% | 100 000 | 1/2 |
| | | CD15− | 97% | 100 000 | 0/1 |
| Ptc+/−p53+/− | 675 | CD15+ | 75% | 100 000 | 1/1 |
| | | CD15− | 99% | 100 000 | 0/2 |
| Ptc+/−p53+/− | 567 | CD15+ | 75% | 35 000 | 1/1 |
| | | CD15− | 96% | 55 000 | 0/1 |
| Ptc+/−p53−/− | 648 | CD15+ | 53% | 50 000 | 1/1 |
| | | CD15− | 95% | 50 000 | 1/2 |
| Ptc+/−p53−/− | 655 | CD15+ | 56% | 30 000 | 1/1 |
| | | CD15− | 99% | 30 000 | 0/2 |
| Ptc+/−p53−/− | 759 | CD15+ | 84% | 20 000 | 1/1 |
| | | CD15− | 99% | 20 000 | 0/3 |

In six of seven injections into mice, including 1^{+/-} MB of all p53 genotypes, CD15⁺ cells were capable of transplanting the disease by 12 weeks, and one of eleven mice injected with equal numbers of CD15⁻ cells showed tumor initiation.

### EXAMPLE 3

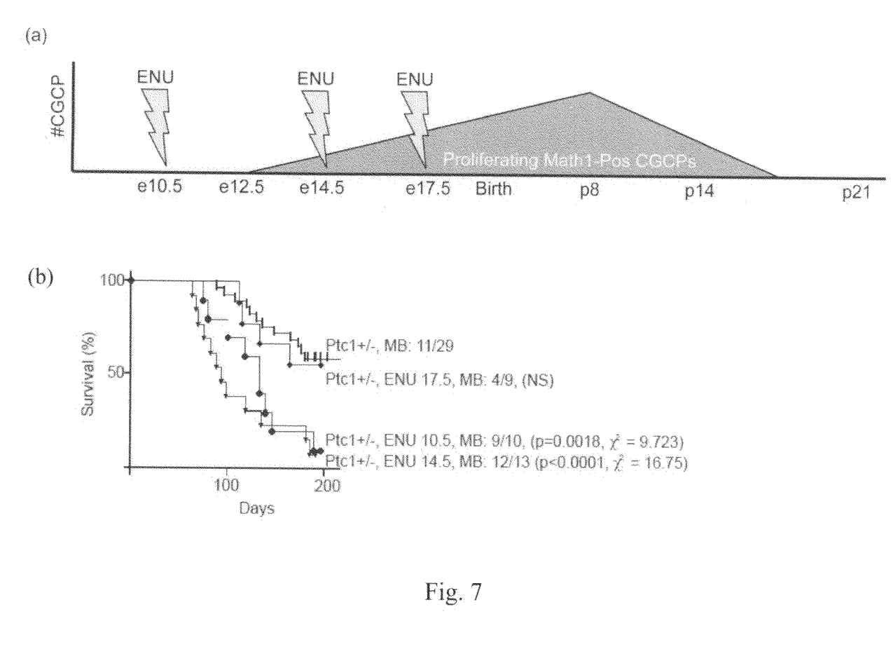

Cerebellar granule cell precursors (CGCPs) are believed to be the cells of origin of medulloblastoma cells, particularly in the Ptc1^{+/-} mouse. Given identification that tumourigenic medulloblastoma cells display similar characteristics to normal neural stem cells, the initiation of medulloblastoma cells from stem cells prior to the birth of Math1⁺ CGCPs was investigated. Ethyl-N-Nitrosoamine (ENU), a chemical carcinogen known to induce multiple tumor types in rats and mice when administered during development, was administered (25 mg/kg ENU) intraperitoneally to timed-pregnant Ptc^{+/-} mice on e10.5, e14.5 or e17.5. Exposed litters were monitored for up to six months (FIG. 7a). ENU administration at e10.5 or e14.5 significantly augmented medulloblastoma incidence to ≧90% ( 9/10 and 12/13 respectively) compared to background ( 11/29) while administration at e17.5 did not ( 4/9) as seen in FIG. 7b. Transplacental ENU treatment did not induce any other tumor type known to arise spontaneously in the Ptc1^{+/-} genotype. The increase of MB incidence in response to carcinogen exposure at early developmental stages, when no CGCPs exist, indicates that medulloblastoma development is initiated from a stem cell.

### Discussion

Thus, a mouse model of medulloblastoma that retains a stem cell hierarchy and is representative of human medulloblastoma disease is herein provided. The cells capable of expansion demonstrate the stem cell properties of self-renewal and multilineage differentiation. Nestin and Sox2 may be used to identify cells that can be expanded in serum free conditions and initiate the formation of heterogeneous medulloblastoma in allogeneic recipients.

Interestingly, Math1 protein expression did not identify a subpopulation in the primary or secondary tumors in vivo, and was expressed in both undifferentiated and differentiated cells in vitro. Recently, it was reported that Math1+ GCPs from normal cerebella and Ptc1^{+/-} tumors differentiate in response to FGF. In contrast, the present results indicate that Nestin+ and Sox2+ cells are enriched when grown in serum free conditions with EGF and FGF. Further, these stem cell markers define the tumourigenic cells within medulloblastoma.

The present observations also demonstrate the tremendous potential that serum free-derived cancer cell lines provide. EGF and FGF culture conditions allow enrichment and expansion of cells that retain stem cell properties, tumour-initiating capacity, and activated developmental signaling pathways in vitro. The present genetic analysis of Ptc1 genotype demonstrates the resemblance of present cell lines to the primary tumour.

We claim:

1. Isolated medulloblastoma-forming clonogenic cells.

2. Isolated cells as defined in claim 1, which are Nestin+Sox2+.

3. Isolated cells as defined in claim 1, which are ptc1-deficient.

4. Isolated cells as defined in claim 1, which are CD15+.

5. Isolated cells as defined in claim 1, which retain at least one of Hedgehog and Notch signaling.

6. An isolated stable medulloblastoma-forming cell line comprising Nestin+Sox2+ medulloblastoma cells.

7. A cell line as defined in claim 6, wherein said cells are ptc1-deficient.

8. A cell line as defined in claim 6, wherein said cells are CD15+.

9. A cell line as defined in claim 6, wherein said cells retain at least one of Hedgehog and Notch signaling.

10. A non-human transgenic animal model transplanted with the cells of claim 1.

11. An animal model as defined in claim 10, wherein the cells possess one or more properties selected from the group consisting of Nestin+Sox2+, ptc1-deficiency, CD15+, activated Hedgehog signaling and activated Notch signaling.

12. A method of producing a cell line as defined in claim 6, comprising the steps of obtaining a sample of medulloblastoma cells and culturing the medulloblastoma cells in serum free conditions with EGF and FGF to establish the cell line.

13. A method as defined in claim 12, wherein the cells possess one or more properties selected from the group consisting of Nestin+Sox2+, ptc1-deficiency, CD15+, activated Hedgehog signaling and activated Notch signaling.

14. A method of screening for candidate therapeutic compounds comprising the steps of culturing a cell line as defined in claim 6 in the presence of a candidate compound for a sufficient period of time and then determining whether the candidate compound inhibits Hh or Notch signaling, wherein inhibition of Hh or Notch signaling is indicative of the therapeutic potential of said compound to treat medulloblastoma.

15. A method as defined in claim 14, wherein the cells possess one or more properties selected from the group consisting of Nestin+Sox2+, ptc1-deficiency, CD15+, activated Hedgehog signaling and activated Notch signaling.

16. A method of screening for candidate therapeutic compounds comprising the steps of administering to an animal model as defined in claim 10 a candidate compounds and determining whether the compound at least reduces the medulloblastoma within the animal, wherein reduction of the medulloblastoma indicates the therapeutic potential of the compound.
